(19) United States
(12) Patent Application Publication
(10) Pub. No.: US 2006/0020257 A1
(43) Pub. Date: Jan. 26, 2006

(54) Retractable intravenous tube organizer

(76) Inventor: Joseph Rolland Mambourg, Ann Arbor, MI (US)

Correspondence Address: Joseph Mambourg, 1690 Waterside Ct., Ann Arbor, MI, 48108, US

(21) Appl. No.: 10/897,542

(22) Filed: Jul. 23, 2004

(51) Int. Cl. A61B 17/00

(52) U.S. Cl. 606/1

(57) ABSTRACT

An intravenous tube organizer for use in hospitals. The present invention consists of a base and a retract base. The base contains a retracting mechanism as a means for retracting and extracting the retract base from the base. The base is attached to the retract base via the retracting mechanism and a cord. Both the base and retract base have an exterior clip that is designed to attach the intravenous tube to the respective bases without the crimping or tearing of the intravenous tube. The ultimate advantage of this device is that it allows for safe organization of intravenous tubes while still allowing the patients full mobility.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION

1. Field of Invention

This invention relates to medical instruments, specifically to a device that organizes an intravenous tube.

2. Discussion of Prior Art

Everyday, hospital patients around the world wake up to a continued fight for their own survival. Modern medicine, with its many technologies and methods, tries to help these individuals as much as possible with the continuous infusion of medicines and other fluids. These injections are usually administered into the patient's body through tubes called Intravenous lines. These Intravenous (I.V.) lines allow the medicine to be injected directly into the blood stream without the need for constant injections. Unfortunately, these lines can only carry one medicine at a time; thus, as many as ten different I.V. lines can be attached to a patient at any one moment. Because of the number of I.V. lines going to a single patient, the I.V. lines can easily tangle and very commonly do. This “spaghetti,” as it is known by the nurses, can be very dangerous for the patients and very time consuming for the nurses to unravel.

Many devices have been created to confront this common problem. Unfortunately, none of these previous devices is capable of solving the multitude of problems associated with the organization of the I.V. lines. The fact is that a new device is needed to solve the entire I.V. line dilemma, not just a segment of it. Without the creation of a new device, I.V. patient care will continue to be unsafe, unorganized, and cluttered.

The first part of the I.V. line dilemma that is not addressed by these previous devices is safety. Many of the previous devices are designed so all the I.V. lines are attached to a single piece of plastic or similar material. This design separates the I.V. lines so they cannot tangle. The problem is that since all the lines are attached to the single piece of plastic, the mobility of the individual I.V. lines is decreased. The reason for this is that since all the lines are attached to a central location, if the patient moves her arm, the I.V. line attached to her leg gets pulled the same distance, potentially pulling either I.V. line out. The requirement of a new device that allows for the safe mobility of both patients and individual I.V. lines is essential.

Similarly, one of the most important dilemmas in the safety of the I.V. line is being able to control the extreme length of the I.V. lines. The average I.V. line is 2.5 meters long and the majority of the previous devices do not reduce this length. This length is needed for mobility but causes many problems for the patients and nurses during transfers and other movement activities. This is because the I.V. lines can be very easily tripped on, an occurrence potentially very harmful for the patient. The necessity of a device that can shorten the I.V. lines while maintaining mobility is essential. This inability of the previous devices to solve the safety dilemma of I.V. lines defines the reason why a new device is needed.

The second part of the I.V. line dilemma that is not addressed by these previous devices is the need to reduce bed side clutter. All of the previous devices require space. Even the simplest devices still require a significant section of the hospital bed when considering the size and nature of current hospital rooms. As the patient's condition improves, the significance of such space becomes even greater since those patients would be expected by the hospital staff to move around. A perfect example of a device that solves a large portion of the I.V. line dilemma but increases the clutter was designed and built by the Biomedical Engineering Department at the University of Wisconsin, Madison¹. This device requires the hospital to attach the device to either the bed rail or to the I.V. bag pole. Such actions would significantly increase the clutter of the hospital room, especially when considering that only one line can be organized per device. This inability of the previous devices to solve the clutter dilemma of I.V. lines also defines the reason why a new device is needed.

In summation, although the previous devices do solve parts of the I.V. line dilemma, they certainly overlook many important segments.

3. Objects and Advantages

Accordingly, several objects and advantages of the present invention are as follows.
a.) To provide a device that reduces the length of the individual intravenous lines.
b.) To provide a device that organizes the intravenous lines.
c.) To provide a device that attaches directly to the intravenous lines for improved mobility.
d.) To provide a device that does not constrict the flow of medicine to the patient.
e.) To provide a device that allows for the extension and retraction of the intravenous lines for increased safety and mobility.

Further objects and advantages are to provide a device that can be attached and detached from the intravenous lines easily, without the crimping or tearing of the intravenous lines, is inexpensive, and is easy to use. Still further objects and advantages will become apparent from a consideration of the ensuing description and drawings.

### SUMMARY

In accordance with the present invention, a Retractable Intravenous Tube Organizer comprises a base having a clip, a clip adjacent to the base clip, and a retractable cord attaching both.

### DRAWINGS—FIGURES

In the drawings, closely related figures have the same number but different alphabetical suffixes.

FIG. 1A shows an expanded isometric view.

FIG. 1B shows an expanded side view.

FIG. 1C shows an expanded top view FIG. 2 shows an isometric view of the present invention.

FIG. 3A shows an isometric view of the retract base.

FIG. 3B shows a top view of the retract base.

FIG. 4 shows an isometric view of the present invention attached to an intravenous line.

FIG. 5 shows an isometric view of the base and base clip with a section of Intravenous line correctly snapped into the base clip.

DRAWINGS-Reference Numerals

10-Attachment Screw
12-Base Bottom
14-Retract Spring
16-Cord Holder
18-Axle
20-Base Top
22a-Retract Clip
22b-Base Clip
24-Cord Holder Rest
26-Spring Attachment Groove
28-Spring Attachment Point
30-Hole
32-Hole
34-Hole
36-Base Assembly
38-Retract Base Assembly
40-Cord
42-Hole
44-Retracting Mechanism Assembly

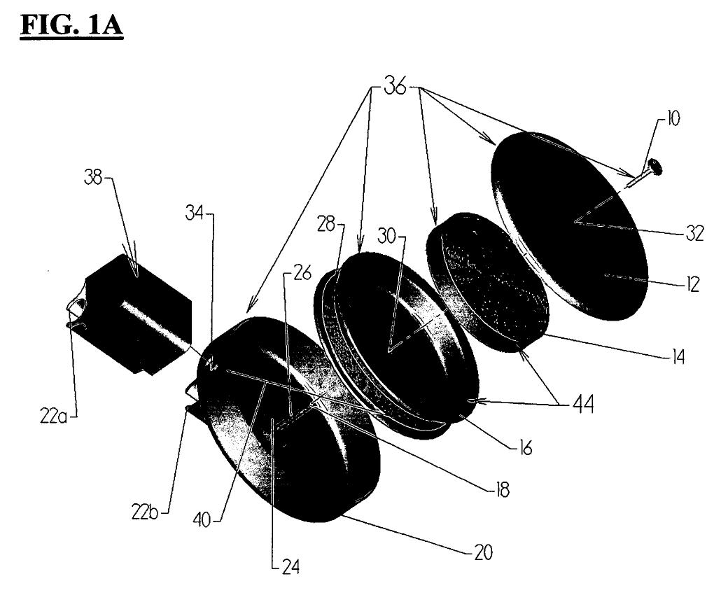

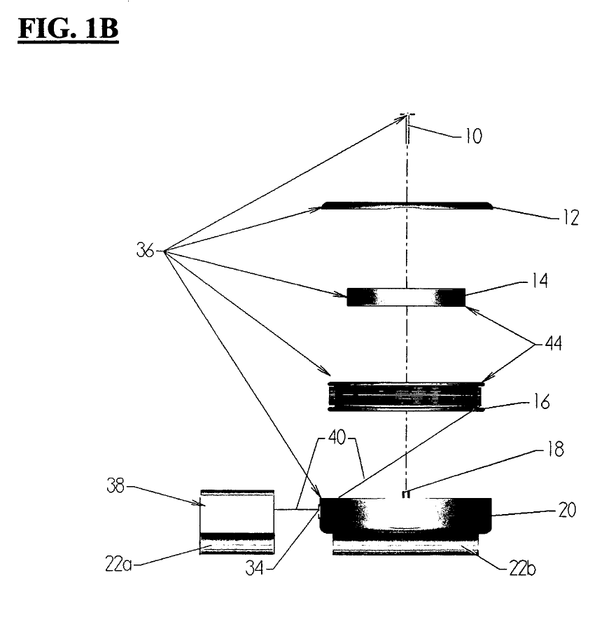

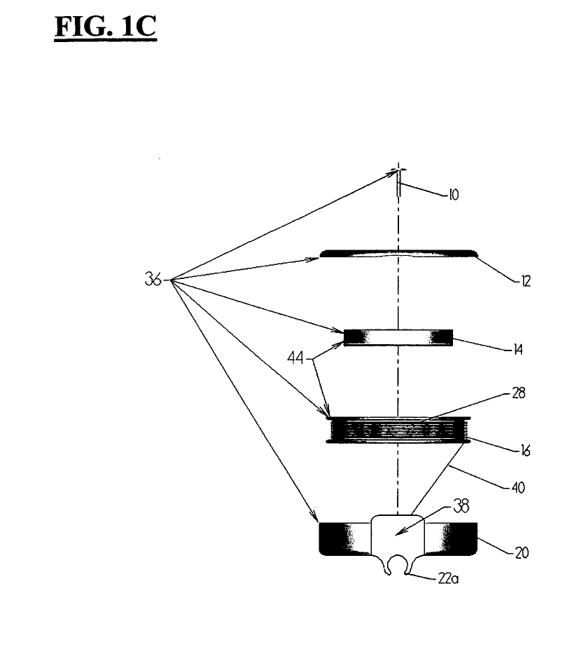

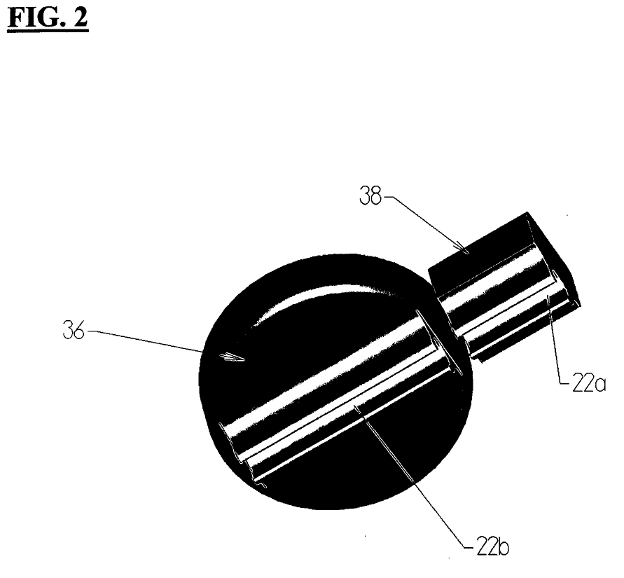

### DETAILED DESCRIPTION—FIGS. 1A, 1B AND 1C—PREFERRED EMBODIMENT

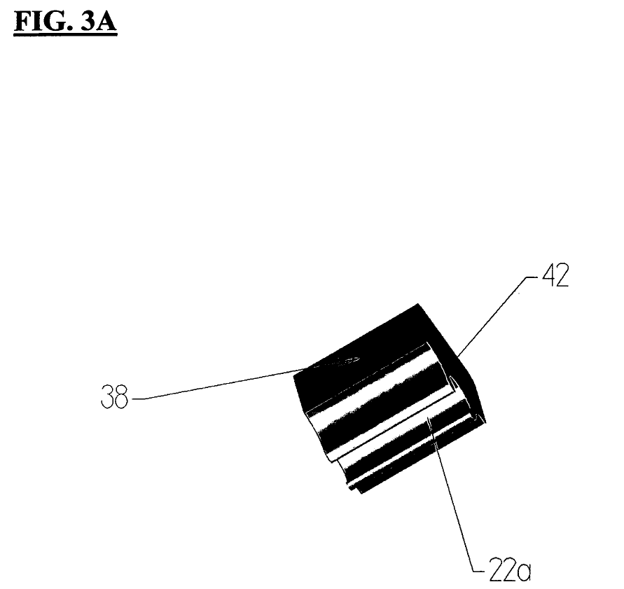

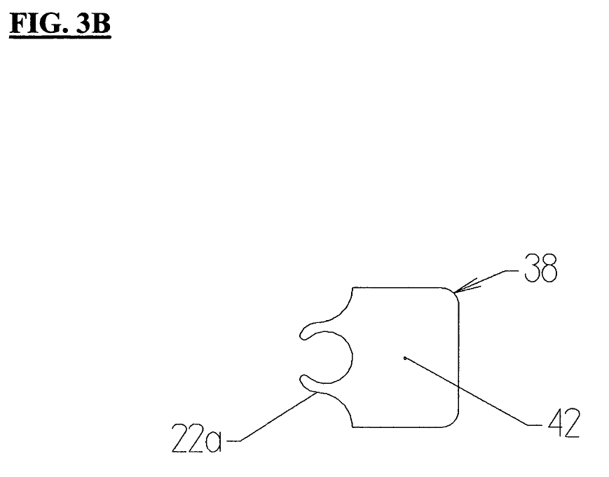

A preferred embodiment of the present invention is illustrated in FIG. 1A (expanded isometric view), FIG. 1B (expanded side view) and FIG. 1C (expanded top view). The present invention has a base assembly 36 consisting of a base clip 22b, a base top 20, a cord holder rest 24, an axle 18, a retracting mechanism assembly 44, a base bottom 12, and an attachment screw 10. The retracting mechanism assembly 44 consists of a cord holder 16 and a retract spring 14. The retract base assembly 38 consists of a retract clip 22a and a hole 42. (FIGS. 3A and 3B.)

In the preferred embodiment of the retracting mechanism assembly 44, the retract spring 14 rests inside the cord holder 16 and is attached to the cord holder at the spring attachment point 28.

In the preferred embodiment of the base assembly 36, the axle 18 is inserted through hole 30 until the retracting mechanism assembly 44 rests on the cord holder rest 24. The retract spring 14 is then inserted through the spring attachment groove 26 which is cut through the center of the axle 18. Both the axle 18 and the cord holder rest 24 are part of the base top 20. The attachment screw 10 is then inserted through the base bottom 12 at hole 32 and into the spring attachment groove 26. The attachment screw 10 is then screwed into the spring attachment groove 26 until tight against the axle 18. The result allows for the rotation of the cord holder 16 around the axle 18 while allowing both the retract spring 14 to function and the base assembly 36 to be fully connected.

In the preferred embodiment, the retract base assembly 38 and the base assembly 36 are connected by a cord 40. First, cord 40 is attached to the retract base assembly 38 at hole 42. Next, cord 40 is inserted into the base assembly 36 through hole 34 and wrapped around the cord holder 16. Finally, cord 40 is attached to the spring attachment point 28. The result allows for the extraction of the retract base assembly 38 from its position next to the base assembly 36 and the automatic retraction of the retract base assembly 38 back to the base assembly 36 with a force that is just greater than the weight of an I.V. line filled with liquid.

In the preferred embodiment, the retract clip 22a is on the same side of the retract base assembly 38 as the base clip 22b.

In the preferred embodiment, the base top 20, the base bottom 12, the retract base assembly 38, the retract clip 22a, cord holder 16 and base clip 22b will be made of any durable plastic. However, any material that is shock resistant could be used.

In the preferred embodiment, both the retract clip 22a and the base clip 22b consist of holes that are drilled to an interference fit with the intravenous line and are opened by a slot that is two thirds the diameter of these holes. However, these clips could be any apparatus of any dimension that would connect the retracting mechanism assembly 44 to the intravenous line without collapsing the I.V. line and restricting flow through the intravenous line.

In the preferred embodiment, the retract spring 14 will be made of non-magnetic stainless steel. However, any material that demonstrates a fixed spring constant over the range of use could be used.

In the preferred embodiment, the base assembly 36 will be held together by a nylon fastener. However, any type of fastener, rivet or adhesive material could be used to attach the base assembly 36. Also, the base assembly 36 could be made of two pieces that snap together to form the required internal structure.

In the preferred embodiment, the cord 40 will be small gauge, nylon string that is approximately three feet in length. However, any type of string or wire could be used at any length.

In the preferred embodiment, the side opposite the base clip 22b will have a surface that would allow nurses to make notes on what type of fluid is flowing through the intravenous tube and to where the intravenous line is attached to the patient.

Finally, in the preferred embodiment the Retractable Intravenous Tube Organizer will be a one time use device. Meaning that after the Retractable Intravenous Tube Organizer is used to organize an individual patient's intravenous line; it will be thrown away for sanitary purposes along with the I.V. line.

Operation—FIG. 1a, 4, 5

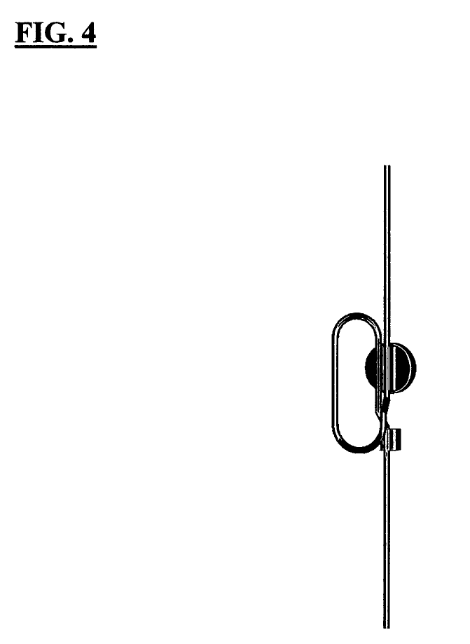

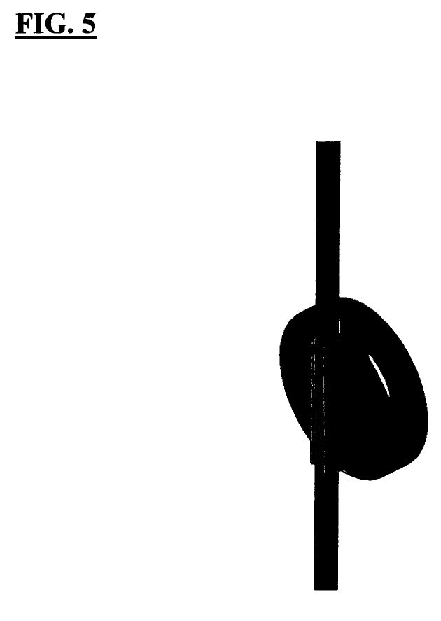

The process of using the Retractable Intravenous Tube Organizer is very simple. One first pushes the base clip 22b onto an intravenous tube at any point on the intravenous tube so the intravenous tube is contained within the base clip 22b as shown in FIG. 5 and the retract base assembly 38 is below the base assembly 36. One then extends the retract base assembly 38 to some point lower on the intravenous tube. Next, one pushes the retract clip 22a onto the intravenous tube at that lower point on the intravenous tube so the intravenous tube is contained within the retract clip as shown in FIG. 5. Finally, one releases the retract base assembly 38, allowing the retract base assembly 38 to retract back to the base assembly 36. The final result of attaching the Retractable Intravenous Tube Organizer is shown in FIG. 4.

Advantages

From the description above, a number of advantages of my Retractable Intravenous Tube Organizer become evident:
(a) The retracting action of the organizer allows for shortening of the intravenous tube.
(b) The shortening of the intravenous tube organizes the intravenous tube into an controlled loop.
(c) The clips allow for simple attachment and detachment of the intravenous line.
(d) The device organizes the intravenous tube while keeping full mobility of the I.V. line for the patient.
(e) The clips attach to the intravenous tube in such a way that will not constrict the flow of medicine to the patient.
(f) The simple design allows for plastic injection molding which reduces the price per unit by a significant margin.
(g) The simple operation of the device allows for quick and uncomplicated usage.
(h) The flat surface of the base bottom allows a place for nurses and other medical employees to label the individual lines with notes on what the tube contains.
(i) The non-magnetic qualities of the organizer also allow for the use of this device in MRI machines without danger to the patient.
(j) The small size allows for increased safety without the increase of hospital room clutter.

### CONCLUSIONS, RAMIFICATIONS, AND SCOPE

Accordingly, the reader will see that the present invention can be used to easily organize an intravenous line, can be easily connected and removed from an intravenous line, and can be easily implemented while still allowing full mobility to the patient. In addition to giving patience full mobility and nurses' quicker access to the correct intravenous tube, the Retractable Intravenous Tube Organizer allows for the complete integration of itself into a hospital atmosphere. Its organizing qualities, small size and simple design help improve patient safety and hospital efficiency.

Although the description above contains many specificities, these should not be construed as limiting the scope of the invention but as merely providing illustrations of some of the presently preferred embodiments of this invention. For example, the base could be many other shapes, such as oval, rectangular, triangular, etc.; the retract base could also have many different shapes; the clips could be an alligator clip or similar device; all the parts of the invention could be made from any colored material; the retract base could be in an infinite number of locations around the base, with the retract clip on any side of the retract base, etc.

Thus the scope of the invention should be determined by the appended claims and their legal equivalents, rather than by the examples given.

I claim:

1. A device for organizing an intravenous tube, comprising of about two attachment clips with the means of attaching said device to an intravenous tube, and a retracting mechanism assembly with a means of decreasing the distance between said attachment clips.
